(19) United States
(12) Patent Application Publication
(10) Pub. No.: US 2010/0010287 A1
(43) Pub. Date: Jan. 14, 2010

(54) Brachytherapy device with one or more toroidal balloons

(75) Inventor: Paul Lubock, Laguna Niguel, CA (US)

Correspondence Address: EDWARD J. LYNCH, PATENT ATTORNEY, FOUR EMBARCADERO CENTER, SUITE 1700, SAN FRANCISCO, CA, 94111, US

(73) Assignee: SenoRx, Inc.

(21) Appl. No.: 12/459,512

(22) Filed: Jul. 2, 2009

Related U.S. Application Data
(60) Provisional application No. 61/134,337, filed on Jul. 9, 2008.

(51) Int. Cl. A61M 36/04

(52) U.S. Cl. 600/7

(57) ABSTRACT

The disclosure is directed to catheter devices and methods for controlled application of irradiation to tissue adjacent a body site, such as cavity after removal of tissue, e.g. cancer. The catheter device includes one or more inflatable, toroidal balloons on the distal shaft section of the device which surrounds and is spaced from one or more radiation guide members. Preferably at least one radiation guide members extend away from a central longitudinal axis to provide asymmetrical irradiation. Connecting members may extend between the radiation guide members and the toroidal balloon.

### RELATED APPLICATIONS

This application is related to provisional application Ser. No. 61/134,337, filed on Jul. 9, 2008, which is incorporated herein in its entirety and which is relied upon for priority.

### FIELD OF THE INVENTION

This invention relates generally to the fields of medical treatment devices and methods of use. In particular, the invention relates to devices and methods for irradiating tissue surrounding a body cavity, such as a site from which cancerous, pre-cancerous, or other tissue has been removed.

### BACKGROUND OF THE INVENTION

In diagnosing and treating certain medical conditions, it is often desirable to perform a biopsy, in which a specimen or sample of tissue is removed for pathological examination, tests and analysis. A biopsy typically results in a biopsy cavity occupying the space formerly occupied by the tissue that was removed. As is known, obtaining a tissue sample by biopsy and the subsequent examination are typically employed in the diagnosis of cancers and other malignant tumors, or to confirm that a suspected lesion or tumor is not malignant. Treatment of cancers identified by biopsy may include subsequent removal of tissue surrounding the biopsy site, leaving an enlarged cavity in the patient's body. Cancerous tissue is often treated by application of radiation, by chemotherapy, or by thermal treatment (e.g., local heating, cryogenic therapy, and other treatments to heat, cool, or freeze tissue).

Cancer treatment may be directed to a natural cavity, or to a cavity in a patient's body from which tissue has been removed, typically following removal of cancerous tissue during a biopsy or surgical procedure. For example, U.S. Pat. No. 6,923,754, U.S. patent application Ser. No. 10/849,410, U.S. patent application Ser. No. 11/593,784 and U.S. patent application Ser. No. 11/716,758, the disclosures of which are all hereby incorporated by reference in their entireties, describe devices for implantation into a cavity resulting from the removal of cancerous tissue which can be used to deliver radiation to surrounding tissue. One form of radiation treatment used to treat cancer near a body cavity remaining following removal of tissue is “brachytherapy” in which a source of radiation is placed near to the site to be treated.

Lubock above describes implantable devices for treating tissue surrounding a cavity left by surgical removal of cancerous or other tissue that includes an inflatable balloon constructed for placement in the cavity. Such devices may be used to apply one or more of radiation therapy, chemotherapy, and thermal therapy to the tissue surrounding the cavity from which the tissue was removed. The delivery lumen of the device may receive a solid or a liquid radiation source. Radiation treatment is applied to tissue adjacent the balloon of the device by placing radioactive material such as radioactive “seeds” in a delivery lumen. Such treatments may be repeated if desired.

U.S. patent application Ser. No. 11/593,784, filed on Nov. 6, 2006, entitled “Asymmetrical Irradiation Device for A Body Cavity” describes a brachytherapy device having a plurality of individual tubular members which extend away from a central longitudinal axis and which are surrounded by an expandable member or balloon. A commercial embodiment of such a device, the Contura™ Multi-Lumen Radiation Balloon (MLB) Catheter, is now being sold by the present assignee SenoRx Inc.

Tissue cavities resulting from biopsy or other surgical procedures such as lumpectomy typically are not always uniform or regular in their sizes and shapes, so that radiation treatment often result in differences in dosages applied to different regions of surrounding tissue, including “hot spots” and regions of relatively low dosage. However, by conforming the tissue lining the cavity about an inflated member, such as a balloon, a more uniform or controlled radiation can be applied to the tissue.

However, making a robust, inflatable balloon which has a predictable inflated size and shape can be problematic, particularly with a balloon size suitable for breast biopsy/lumpectomy cavities which range from about 0.5 to about 4 inches in maximum diameter, and are typically about 2 inches.

### SUMMARY OF THE INVENTION

The present invention is directed to a brachytherapy catheter having an elongated shaft, a distal shaft section, one or more toroidal shaped expandable. members surrounding a length of the distal shaft section and at least one radiation guide member having an inner guide or lumen for locating a radiation source along the length of the distal shaft section. Preferably, the one or more toroidal balloons are shorter than the length of the distal shaft section. The one or more balloons are secured to the balloon catheter, preferably to one or more of the radiation guide members or to the distal tip of the catheter. The connections may be stiff or flexible but the assembly must be suitable for advancement through a patient's tissue with minimal difficulty.

### BRIEF DESCRIPTION OF THE DRAWINGS

FIG. 1 is a perspective view of a catheter embodying features of the invention illustrating a toroidal balloon in an inflated condition surrounding the guide members which receive the radiation source.

FIG. 2 is a longitudinal cross-sectional view of the distal shaft section of the catheter taken along the lines 2-2 shown in FIG. 1.

FIG. 3 is a transverse cross-section view of the catheter as shown in FIG. 2 taken along the lines 3-3 shown in FIG. 2.

FIG. 4 is a longitudinal cross-sectional view similar to that shown in FIG. 1 with a different connection between the toroidal balloon and the catheter shaft.

FIGS. 5A-5C are schematic transverse cross-sectional views of alternative toroidal balloon shapes and sizes.

### DETAILED DESCRIPTION OF EMBODIMENTS OF THE INVENTION

The present invention provides devices and methods for treatment of a patient's body cavity. Specifically, devices and methods having features of the invention may used to deliver radiation into a biopsy site or into a cavity left after removal of cancerous tissue from the patient's body.

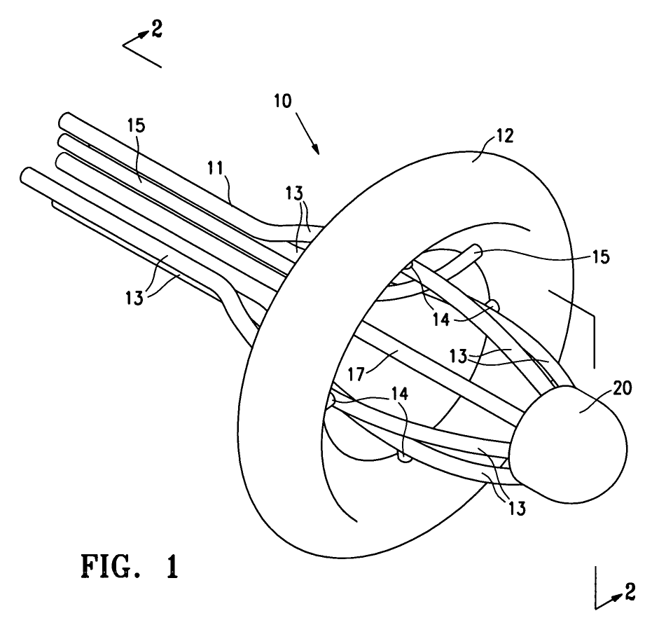

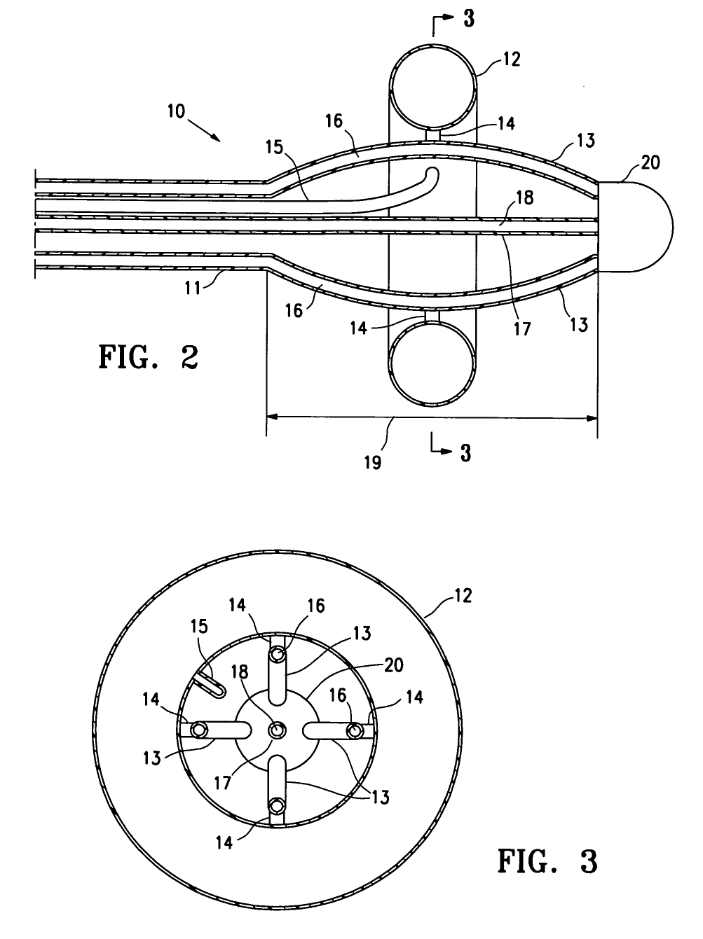

FIGS. 1-3 illustrate a brachytherapy balloon catheter 10 embodying features of the invention which has a distal shaft section 11 surrounded by toroidal balloon 12. A plurality of individual tubular radiation guide members 13 extend along the length of the distal shaft section 11. The toroidal balloon 12 is secured to individual tubular radiation guide members 13 by means of connection elements 14. The balloon 12 is filled with inflation fluid through tubular inflation member 15 which is in fluid communication with the interior of the balloon 12. The tubular guide members 13 have inner guides or lumens 16 which receive a radiation source such as a radioactive pellet (not shown) and guide the radiation source to a desired position within the guide along the distal shaft section 11. The connections 14 may be an extension of the balloon or tubular members 12 or they may be a separate member or adhesive. The central tubular radiation guide member 17 is provided with an inner guide or lumen 18 which may also receive a radiation source during the treatment and guide the source to a desired location therein along the length 19 of the distal shaft section 11. Further details of the catheter may be found in the above referenced application which is incorporated herein by reference. The length of the distal shaft section 18 in which the radiation source may be located is indicated by line 19. The distal ends of the tubular guide members 14 and 16 are secured to the distal tip 20 at the distal end of the catheter. The tubular guide members may be formed strong enough to provide support to the distal shaft section 11 of the catheter as it is advanced within the patient. Alternatively, one or more longitudinal support members (not shown) may be provided that extend along at least the distal shaft section and have distal ends secured to the distal tip 20. Vacuum lines may extend into the distal shaft section to withdraw gas, fluid and/or debris from the treatment cavity. While only one toroidal balloon 12 is shown in the drawing, the distal shaft section may be provided with a plurality of such balloons along the length of the treatment location. Such plurality of toroidal shaped balloons may be longitudinally spaced apart or may be in contact with one another. Moreover, a plurality of toroidal balloons may be secured together to form the expandable member.

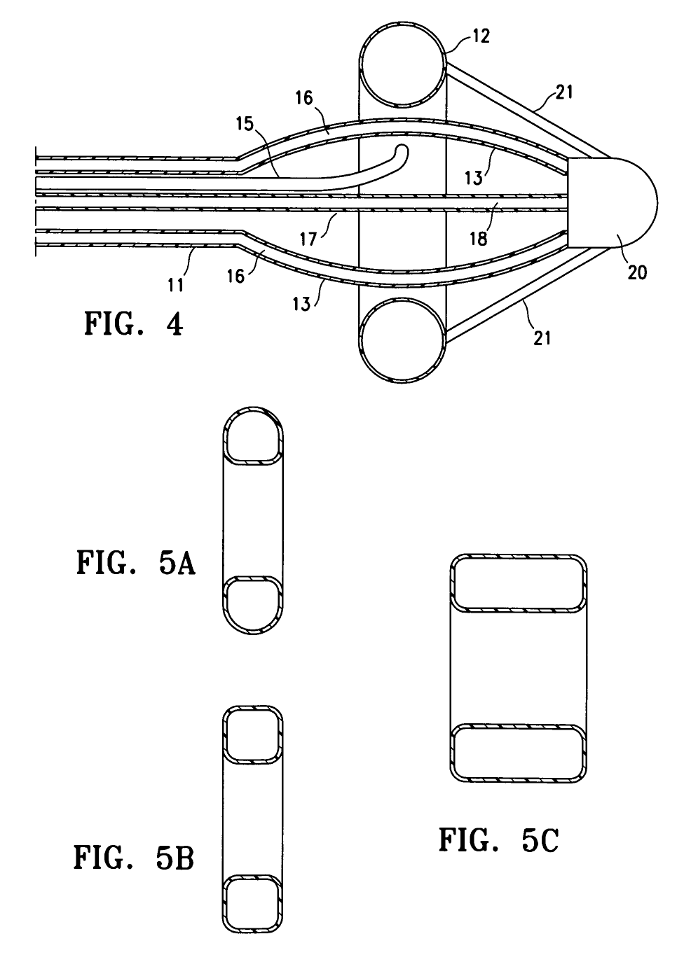

FIG. 4 illustrates an alternative construction for connecting the balloon 12 to the catheter. In this embodiment the connecting members 21 secure the balloon 12 to the distal tip 20. The connecting members 21 are preferably flexible enough in the longitudinal direction so that they lay against the distal shaft section 11 when advanced to the biopsy site as described in the aforesaid application.

FIGS. 5A-5C represent alternative balloon shapes and sizes that may be employed with the catheter of the invention. In FIG. 5A, the balloon has a flat inner surface and a rounded outer surface. FIG. 5B illustrates a balloon with a square transverse cross-section. FIG. 5C illustrates a balloon with a rectangular transverse cross-section.

The plurality of tubular guide members 13 and 17 may extend proximally to an adapter (not shown) on the proximal end of the catheter. Alternatively, the catheter 10 may have a proximal shaft section (not shown) having a plurality of lumens which are in fluid communication with the lumens in the tubular guides located in the distal shaft section and which are configured to receive one or more radiation sources and guide the sources to the inner lumens 16 and 18 of the tubular guide members 12 and 16. These features are shown in copending applications Ser. No. 11/593,784 and Ser. No. 11/716,758.

The tubular members 12 and 16 may have a support element shown in copending applications (Ser. No. 11/593,784 and Ser. No. 11/716,758), which would extend along length of the treatment location and have compartments which are designed to receive and support the tubular radiation guide members.

All of the tubular guide members which extend through the treatment location would not necessarily be used in a particular irradiation procedure, but they would be available for use by the physician if needed, e.g. when the balloon 12 of the radiation catheter 10 is not in a desired position and rotation of the catheter is not appropriate or desirable.

The radiation source for the brachytherapy device is preferably a radiation seed on the distal end of rod, but the radiation source may be a solid or liquid radiation source. Solid radionuclides suitable for use with a device 10 embodying features of the present invention are currently generally available as brachytherapy radiation sources available from Med-Tec, Orange City, Iowa. Suitable liquid radiation sources include, for example, a liquid containing a radioactive iodine isotope (e.g., I¹²⁵ or I¹³¹), a slurry of a solid isotope, for example, ¹⁹⁸Au or ¹⁶⁹Yb, or a gel containing a radioactive isotope. The radiation source is usually loaded into the catheter device 10 after placement into a body cavity or other site of a patient.

The various device components can be provided, at least in part, with a lubricious coating, such as a hydrophilic material. The lubricious coating preferably is applied to the elongate shaft or tubular members and to the toroidal balloon, to reduce sticking and friction during insertion and withdrawal of the device 10. Hydrophilic coatings such as those provided by AST, Surmodics, TUA Systems, Hydromer, or STS Biopolymers are suitable. The surfaces of the device 10 may also include an antimicrobial coating that covers all or a portion of the device 10 to minimize the risk of introducing of an infection during extended treatments. The antimicrobial coating preferably is comprised of silver ions impregnated into a hydrophilic carrier. Alternatively the silver ions are implanted onto the surface of the device 10 by ion beam deposition. The antimicrobial coating may also be an antiseptic or disinfectant such as chlorhexadiene, benzyl chloride or other suitable biocompatible antimicrobial materials impregnated into hydrophilic coatings. Antimicrobial coatings such as those provided by Spire, AST, Algon, Surfacine, Ion Fusion, or Bacterin International would be suitable. Alternatively a cuff member covered with the antimicrobial coating may be provided on the elongated shaft of the delivery device 10 at the point where the device 10 enters the patient's skin.

The balloon 11 may also be filled with radiopaque inflation material to facilitate detection during CT, X-ray or fluoroscopic imaging. Such imaging allows the physician or other staff to detect the size and shape of the balloon and whether the balloon is properly located at the desired location. Alternatively, the exterior surface of an inner layer of the balloon may be coated at least in part with radiopaque material.

The device 10 may be used to treat a body cavity of a patient, e.g. a biopsy or lumpectomy site within a patient's breast, in the manner described in the previously referred to co-pending applications. Usually the proximal end of the catheter device extends out of the patient during the procedure when the balloon is inflated.

Radiation balloon catheters for breast implantation generally are about 6 to about 12 inches (15.2-30.5 cm) in length, typically about 10.6 inch (27 cm). The shaft diameter is about 0.1 to about 0.5 inch (2.5-12.7 mm), preferably about 0.2 to about 0.4 inch (5.1-10.2 mm), typically 0.32 inch (8 mm). The individual radiation lumens are about 0.02 to about 0.15 inch (0.5-3.8 mm), preferably about 0.04 to about 0.1 inch (1-1.5 mm). The balloons are designed for inflated configurations about 0.5 to about 4 inches (1.3-10.2 cm), typically about 1 to about 3 inches (2.5-7.5 cm) in transverse dimensions, e.g. outer diameters.

While particular forms of the invention have been illustrated and described herein, it will be apparent that various modifications and improvements can be made to the invention. To the extent not previously described, the various elements of the catheter device may be made from conventional materials used in similar devices. Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. All patents and all patent applications referred to above are hereby incorporated by reference in their entirety. Further details of brachytherapy catheters can be found in the patents and applications incorporated herein by reference.

Terms such as “element”, “member”, “component”, “device”, “means”, “portion”, “section”, “steps” and words of similar import when used herein shall not be construed as invoking the provisions of 35 U.S.C §112(6) unless the following claims expressly use the terms “means for” or “step for” followed by a particular function without reference to a specific structure or a specific action.

What is claimed is:

1. A brachytherapy device comprising:
a. elongated shaft having a distal shaft section and a distal tip distal to the distal shaft section;
b. at least one radiation guide member extending along a length of the distal shaft section having an inner guide which is configured to receive a radiation source and facilitate advancement of the radiation source within the inner guide; and
c. at least one toroidal shaped expandable member disposed about the radiation guide member.

2. The brachytherapy device of claim 1 wherein the expandable member is a balloon.

3. The brachytherapy device of claim 2 wherein the balloon is secured to one or more of the radiation guide members.

4. The brachytherapy device of claim 2 wherein the balloon is secured to the distal tip.

5. The brachytherapy device of claim 6 wherein the balloon is secured to the distal tip by connecting members or struts.

6. The brachytherapy device of claim 1 including a plurality of radiation guide members extending along the length of the distal shaft section.

7. The brachytherapy device of claim 1 including at least one radiation guide member extends away from a central longitudinal axis to provide asymmetrical irradiation when a radiation source is disposed within the radiation guide member.

8. The brachytherapy device of claim 7 wherein the at least one radiation guide member is a tubular member.

9. The brachytherapy device of claim 1 wherein a plurality of toroidal expandable members are provided on the distal shaft section.

10. A method of irradiating tissue surrounding a body cavity, comprising:
a. providing a brachytherapy device which has an elongated shaft with a distal shaft section, a plurality of arcuate tubular members disposed about a length of the distal shaft section and at least one toroidal shaped expandable member which is disposed about the length of the distal shaft section and which has an outer surface spaced from the arcuate tubular members;
b. advancing the brachytherapy device within a patient's body until the distal shaft section is located within the body cavity;
c. expanding the at least one toroidal expandable member within the body cavity; and
d. advancing a radiation source through at least one of the radiation guide members to the length of the distal shaft section to irradiate tissue surrounding the body cavity.
